(19) United States
(12) Patent Application Publication
(10) Pub. No.: US 2012/0046226 A1
(43) Pub. Date: Feb. 23, 2012

(54) Combined Treatment of Multiple Sclerosis

(75) Inventors: Alain Creange, Montgeron (FR); Frédéric Galacteros, Santeny (FR); Jean-Pascal Lefaucheur, Paris (FR)

(73) Assignee: Assis-tance Publique - Hopitaux de Paris, Paris (FR)

(21) Appl. No.: 13/148,626

(22) PCT Filed: Feb. 12, 2010
(86) PCT No.: PCT/IB10/00426

§ 371 Date: Sep. 29, 2011

(30) Foreign Application Priority Data
Feb. 12, 2009 (IB) 2009/000481

(51) Int. Cl. A61K 38/18; A61K 31/16; A61P 25/00; A61K 31/195; A61K 31/4439; A61K 31/4412; A61K 31/426

(52) U.S. Cl. 514/7.7; 514/348; 514/616; 514/365; 514/566; 514/333

(57) ABSTRACT

The invention concerns a novel treatment multiple sclerosis, based on induced anaemia, followed by administration of an erythropoiesis-stimulating agent (ESA). In a preferred embodiment, the ESA is darbopoietin alpha, CERA or Hematide. The anaemia is induced by successive bloodlettings or by administration of an iron chelator, such as deferiprone, deferoxamine, polyanionic amines, substituted polyaza compounds, desferrithiocon, hydroxybenzyl-ethylenediamine-diacetic acid and pyridoxal isonicotinoyl hydra z one.

The present invention pertains to the treatment of multiple sclerosis and other related neuropathies.

Multiple sclerosis (MS) is an inflammatory demyelinating disease of the central nervous system that affects at least 2.5 million people worldwide. It is approximately twice as common among women as men. Worldwide, its prevalence varies geographically. Prevalence is highest in countries distant from the Equator, for instance Scotland and Scandinavia. Peak incidence is within the third and fourth decades; it is extremely uncommon to make a new diagnosis in patients over the age of 60 years. There are four main varieties of multiple sclerosis, as defined in an international survey of neurologists (Lublin and Reingold, 1996):

1. Relapsing/Remitting (RRMS) is characterised by relapses (also known as exacerbations) during which new symptoms can appear and old ones resurface or worsen. The relapses are followed by periods of remission, during which the person fully or partially recovers from the deficits acquired during the relapse. Relapses can last for days, weeks or months and recovery can be slow and gradual or almost instantaneous.

2. Secondary Progressive (SPMS) often appears after a number of years of relapsing/remitting MS. This is characterised by a gradual worsening of the disease between relapses.

3. Progressive Relapsing Multiple Sclerosis (PRMS): this form of MS follows a progressive course from onset, punctuated by relapses. There is significant recovery immediately following a relapse but between relapses there is a gradual worsening of symptoms.

4. Primary Progressive (PPMS) is characterised by a gradual progression of the disease from its onset with no remissions at all.

The predominant concept of the natural history of multiple sclerosis (MS) combines two phenomena that support the existence of two disease phases, i.e., the remitting phase as a consequence of the inflammatory process, and the progressive phase related to a neurodegenerative process. Pharmacological treatments of MS essentially target the inflammatory component of the disease and mainly act by reducing the handicap related to the relapses. No disease-modifying drugs have been shown to have any effect on the natural course of the progressive phase. Interestingly, it has recently been suggested that the progression of MS might be directly related to the emergence of a degenerative process that is independent from the inflammatory component of the disease. This degenerative process is directly related to the consequence of demyelination and therefore begins in the early months of the relapsing-remitting phase.

Secondary progressive multiple sclerosis is now considered as an axonal degenerative process related to the consequences of demyelination.

Demyelination induces morphological and physiological changes in the physiology of influx propagation. One of the major consequences is the requirement of additional energy from ATP degradation into ADP in the mitochondria, in order to extrude Na+ after depolarization and maintain transmembrane ion gradients during a continuous conduction. This additional energy is required by the continuous conduction and altered capacity of the myelin sheath resulting from demyelination.

Originally known as a haematopoietic growth factor, erythropoietin (EPO) has been shown to have neuroprotective properties (Ehrenreich et al., 2004; Genc et al., 2004) and promotes neurite outgrowth and axonal repair, neurogenesis and angiogenesis (Bianchi et al., 2004; Toth et al., 2008). It also has anti-apoptotic and anti-oxidative properties (Genc et al., 2006; Keswani et al., 2004; Siren et al., 2001; Wiese et al., 2008).

These properties have been shown to be involved in different pathophysiological processes including traumatic, degenerative and inflammatory disorders such as experimental autoimmune encephalitis (EAE) (Adembri et al., 2008; Boesch et al., 2008; Hartley et al., 2008; Li et al., 2004). A few open trials have been conducted in patients with stroke (Ehrenreich et al., 2002), critically ill patients (Corwin et al., 2007) and multiple sclerosis (Ehrenreich et al., 2007). These trials raise interesting therapeutic options.

It has recently been shown that iron metabolism could be altered in patients with multiple sclerosis (Abo-Krysha and Rashed, 2008), supporting the persistence of an inflammatory process. Excess of iron may have several consequences for the axon, including iron catalyzed production of free radicals that in turn cause oxidative tissue injury. Iron deposits have been observed within some neurons of MS patients (Forge et al., 1998). Moreover, iron accumulation is deleterious for oligodendrocytes physiology (Khwaja and Volpe, 2008).

Despite raising new opportunities, treatment options based on iron deprivation have been disappointing in previous human investigations. Indeed, although iron chelation has given interesting results in an experimental model of multiple sclerosis (Mitchell et al., 2007), human investigation has not been associated with improvements of clinical performance (Lynch et al., 1996).

Treatment of MS with erythropoietin has only been tested in a few patients in an open series (Ehrenreich et al., 2007). Although high dosage of this drug has been associated with improvements in maximum walking capacities, erythropoietin is of limited usefulness in patients without anaemia, considering the risk of thrombosis when haemoglobin is higher than 13 gram per litre (Bokemeyer et al., 2007).

In this context, the inventors have investigated a novel treatment of neurodegenerative diseases with an inflammatory component, in particular multiple sclerosis. This treatment is based on induced anaemia, followed by administration of exogenous erythropoietin. Surprisingly, they have observed a synergistic effect of the two components of this treatment. Indeed, iron deprivation until induction of anaemia resulted in production of endogenous erythropoietin and improvement of cortical excitability (and in some cases of clinical scales), and the subsequent administration of exogenous erythropoietin proved to be well tolerated and lead to clinical results which were better than those previously described (Ehrenreich et al., 2007).

The present invention hence pertains to the use of an erythropoiesis-stimulating agent, for the preparation of a composition for treating multiple sclerosis or a chronic inflammatory demyelinating polyneuropathy in a patient depleted in iron. In what follows, a “patient depleted in iron” designates a patient having an anaemia, whatever the cause of this anaemia. For example, this anaemia can have been induced by successive bloodlettings, as described in the experimental part below, or by the administration of an iron chelator to said patient. Of course, patients who are spontaneously anaemic are considered as “depleted in iron” in the sense of the present invention. In the present text, a patient will be considered as “depleted in iron”, or “in an anaemic state” if the haemoglobin and the ferritin levels, as well as the MCV of said patient are as follows:

Haemoglobin level ≦12.5 g/l in male
≦11.5 g/l in female,

Ferritin level ≦50 μg/l, and

Mean Corpuscular Volume (MCV) <82 femtolitres.

The erythropoiesis-stimulating agent (ESA) used in the compositions according to the present invention can be of any origin and any chemical nature, provided it is compatible with therapeutic administration. In a preferred embodiment, this ESA is recombinant human erythropoietin (rhEPO), such as that commercialized by Roche™ under the reference Neoreconuon®. Purified erythropoietin from human samples can also be used.

Alternatively, an erythropoiesis-stimulating agent distinct from erythropoietin can be used instead of EPO itself. Such ESAs can be protein-based mimetics or agonists, or small molecules. Small molecules which can be used as ESAs according to the present invention can be either peptidic such as Hematide, which is a pegylated synthetic dimeric peptide, or non-peptidic, such as the molecule described by Qureshi et al. (Qureshi et al., 1999). Examples of ESAs which can be used instead of EPO to perform the present invention are cited in (Macdougall, 2008), especially in Table 1. In a preferred embodiment, the invention can be performed with an ESA selected in the group consisting of darbopoietin alpha, CERA and Hematide.

Of course, a nucleic acid designed to cause the expression of erythropoietin or of another ESA can be used instead of the protein. By “designed to cause the expression of . . . ” is herein meant that the considered protein is expressed when the nucleic acid is introduced in an appropriate cell. The region encoding the ESA will typically be situated in the polynucleotide under control of a suitable promoter (such as a strong constitutive, or an endogenous promoter). A nucleic acid according to the invention can be administered directly, or using an appropriate vector. Suitable vector systems include naked DNA plasmids, liposomal compositions to enhance delivery, and viral vectors that cause transient expression. Non-limitative examples of viral vectors are adenovirus or vaccinia virus vectors and vectors of the herpes family, especially in a non-replicative form.

Since, for the purpose of the present invention, EPO and ESAs different from EPO, as well as nucleic acids encoding them, are equivalent (except concerning the posology and sometimes the route of administration), the fact that such a molecule can be used instead of EPO will not be systematically recalled. In case an ESA is used instead of EPO, the physician will adapt the dosage and the frequency and route of administration to obtain the equivalent of the dosages indicated hereafter for rhEPO.

The present invention also pertains to an iron chelator, for use as a medicament for inducing an anaemic state through iron deprivation, in a patient suffering from multiple sclerosis or from a chronic inflammatory demyelinating polyneuropathy. In particular, this anaemic state is induced according to the invention to increase the production of endogenous EPO in said patient, thereby improving the patient's condition, and to prepare said patient for receiving exogenous erythropoietin such as rhEPO.

As described in the experimental part below, once an anaemic state is obtained in a patient suffering from multiple sclerosis, exogenous EPO can advantageously be administered to said patient, since the combination of both treatments leads to synergistic effects improving the patient's condition.

When long-term treatment is wanted, it can be necessary to maintain the anaemic state of said patient by combining the ESA treatment with the administration of an iron chelator. The ESA and the iron chelator can be administered either simultaneously or sequentially, not necessarily at the same frequency, depending on the patient's response.

A further aspect of the present invention is hence an iron chelator, for use as a medicament for treating multiple sclerosis or a chronic inflammatory demyelinating polyneuropathy in a patient also receiving an erythropoiesis-stimulating agent such as rhEPO. According to this aspect of the invention, a “patient also receiving an ESA” is a patient who regularly receives an ESA, at a variable frequency for at least three months, with a minimum dosage equivalent to 20,000 UI of rhEPO per week during this period of time.

The present invention also pertains to the use of an ESA and an iron chelator for the preparation of a composition for treating multiple sclerosis; preferably, said iron chelator is administered to render or maintain a patient depleted in iron. In particular embodiments, said ESA is administered to a patient depleted in iron and/or said iron chelator is administered before said ESA.

Numerous iron chelators have been described in the literature. According to the observed binding to iron, iron chelators may be classified into bidentate, tridentate or hexadentate chelators.

Specific bidentate iron chelators comprise 1,2-dimethyl-3-hydroxypyridin-4-one (Deferiprone, DFP or Ferriprox) and 2-deoxy-2-(N-carbamoylmethyl-[N′-2′-methyl-3′-hydroxypyridin-4′-one])-D-glucopyranose (Feralex-G).

Specific tridentate iron chelators comprise pyridoxal isonicotinyl hydrazone (PIH), 4,5-dihydro-2-(2,4-dihydroxyphenyl)-4-methylthiazole-4-carboxylic acid (GT56-252), 4,5-dihydro-2-(3′-hydroxypyridin-2′-yl)-4-methylthiazole-4-carboxylic acid (desferrithiocin or DFT) and 4-[3,5-bis(2-hydroxyphenyl)-[1,2,4-]triazol-1-yl]benzoic acid (deferasirox). Substituted 3,5-diphenyl-1,2,4-triazoles, e.g., 4-[3,5-bis(2-hydroxyphenyl)-[1,2,4]triazol-1-yl]benzoic acid (deferasirox), their process of manufacture and use thereof are disclosed in the international patent application WO 97/49395. An advantageous pharmaceutical preparation of such compounds in the form of dispersible tablets is disclosed in the international patent application WO 2004/035026.

Specific hexadentate iron chelators comprise N,N′-bis(o-hydroxybenzyl) ethylenediamine-N,N′-diacetic acid (HBED), N-(5-C3-L (5-aminopentyl)hydroxycarbamoyl)-propionamido)pentyl)-3(5-(N-hydroxyacetoamido)-pentyl)carbamoyl)-proprionhydroxamic acid (deferoxamine, desferoxamine or DFO) and hydroxymethyl-starch-bound deferoxamine (S-DFO). Further derivatives of DFO include aliphatic, aromatic, succinic and methylsulphonic analogs of DFO and specifically, sulfonamide-deferoxamine, acetamide-deferoxamine, propylamide deferoxamine, butylamide-deferoxamine, benzoylamide-deferoxamine, succinamide-derferoxamine and methylsulfonamide-deferoxamine.

A further class of iron chelators is the biomimetic class, such as those described by Meijler et al. (Meijler et al., 2002). Certain substituted 3,5-diphenyl-1,2,4-triazoles also have valuable pharmacological properties for iron chelation (Bergeron et al., 1991). Other iron chelators which can be used according to the invention are polyanionic amines, substituted polyaza compounds and desferrithiocon.

Any of the above iron chelators can be used according to the invention, as well as any other iron chelator not mentioned in the above list. However, in a preferred embodiment, the iron chelator used according to the present invention is selected in the group consisting of deferiprone, deferoxamine, deferasirox, polyanionic amines, substituted polyaza compounds, desferrithiocon, hydroxybenzyl-ethylenediamine-diacetic acid and pyridoxal isonicotinoyl hydrazone.

The present invention also pertains to a kit of parts comprising, in separate containers or vials, an ESA and an iron chelator. In such a kit, the ESA and the iron chelator are as described above. Each component can be formulated for direct administration (through any route), for example in pills, caplets, capsules, tablets, powder, cream, syrup, suppository, ointment etc. Alternatively, the components of the kit can be in a form which necessitates a preparation before administration (for example, in a lyophilized form which necessitates suspension in a liquid for subcutaneous (SC) or intravenous (IV) administration).

A kit according to the present invention can also contain a notice of use which indicates at least that the kit can be used for the treatment of multiple sclerosis. This notice can also describe the posology for each of the components.

Another aspect of the present invention is a pharmaceutical composition comprising an ESA and an iron chelator. Such a composition can be formulated to be administered via subcutaneous or intravenous routes, but can also be formulated for oral administration, when appropriate iron chelator and ESA are used.

The present invention will be more fully understood by reading the experimental part which follows, in which the inventors describe the results obtained by a method for treating multiple sclerosis in a patient, comprising the steps of:

1—inducing anemia in said patient, and

2—administrating EPO to said patient.

In the description which follows, step 1 has been performed by successive bloodlettings, but the skilled artisan can easily replace or complete bloodlettings by the use of an iron chelator.

The skilled artisan can also modify step 2 by replacing (either partially or totally) exogenous erythropoietin by another erythropoiesis-stimulating agent.

### EXPERIMENTAL RESULTS

The inventors have investigated a new treatment modality of SPMS which combines iron deprivation and administration of exogenous erythropoietin, in a two-steps approach:
iron deprivation, through bloodletting, may induce improvement through two processes: (i) it decreases the inflammatory process and modifies the distribution of iron in the inflammatory cells; (ii) the artificially induced anaemia resulting from iron deprivation secondary to bloodletting increases the production of endogenous erythropoietin.
subsequent treatment by erythropoietin increases the neuroprotective effect of this molecule on the nervous system receptors, and, in a context of iron depletion, erythropoiesis is not efficacious, precluding any risk of thrombosis related to increase in the concentration of haemoglobin.

The inventors have made the hypothesis that this double steps approach could improve the physiology of the central nervous system through increase of axonal energy availability from NA+/K+ ATPase pump and sodium gradients dependant physiology, supporting this approach as a disease modifying approach of secondary progressive multiple sclerosis and as well as other neurodegenerative disorders with similar mechanisms of degeneration, including relapsing remitting multiple sclerosis (RRMS), primary progressive multiple sclerosis (PPMS), Progressive Relapsing Multiple Sclerosis (PRMS), transitional forms of MS (one relapse followed several years later by a progressive course), as well as inflammatory and/or demyelinating peripheral neuropathies, Parkinson's disease, Alzheimer's disease, Huntington's disease, and diabetic neuropathy.

Objective

Primary: To demonstrate the efficacy of iron deficiency obtained through repeated bloodletting, followed by the administration of recombinant human erythropoietin alpha (rhEPO) on central nervous system function in patients suffering from multiple sclerosis.

Secondary: (1) To evaluate the effect of the combination of iron deficiency and recombinant human erythropoietin alpha on clinical scores in patients suffering from multiple sclerosis. (2) To study the tolerability and the safety of the combination of iron deficiency and recombinant human erythropoietin alpha

Study

Monocentric, Open Study. Total observation period 48 weeks.

Sample Size

Four patients.

Patients and Methods

Patients

Four patients were included. They fulfilled the diagnosis of secondary multiple sclerosis. Clinical features are reported in tables 1a, 1b, 1c and 1d. All four patients have been in the progressive phase of multiple sclerosis for more than 5 years.

Table 1E synthesizes the several treatment phases for each patient.

TABLE 1a
Bloodletting and treatment on patient PER

| | | bloodletting | EPO | EPO | | | |
|---|---|---|---|---|---|---|---|
| patient | Dates | 200 ml | 20000UI/sem | 30000UI/sem | fatigue | crampes | Blood pressure |
| PER | 23/04/2008 | 1 | | | * | | |
| | 30/04/2008 | 1 | | | | | |
| | 07/05/2008 | 1 | | | | | |
| | 14/05/2008 | 1 | | | | | |
| | 29/05/2008 | 1 | | | | | |
| | 12/06/2008 | 1 | | | | * | |
| | 24/06/2008 | 1 | | | | | |
| | 24/07/2008 | 1 | | | | | |
| | 25/08/2008 | 1 | | | | * | |
| | 12/09/2008 | | 1 | | | * | 15/09 |
| | 19/09/2008 | | 1 | | | | 13/09 |
| | 26/09/2008 | | 1 | | | | 14.5/09 |
| | 03/10/2008 | | 1 | | | * | 12/08 |
| | 10/10/2008 | | 1 | | | | 14/08 |
| | 17/10/2008 | | 1 | | | | |
| | 24/10/2008 | | 1 | | | | |
| | 31/10/2008 | | 1 | | | | |
| | 07/11/2008 | | 1 | | | | |
| | 14/11/2008 | | 1 | | | | |
| | 21/11/2008 | | 1 | | | | |
| | 28/11/2008 | | 1 | | | | |
| | 05/12/2008 | | 1 | | | | |
| | 10/12/2008 | | | | | | 14/08 |
| | 12/12/2008 | | 1 | | | | |
| | 15/12/2008 | | 1 | | | | |

TABLE 1b
Bloodletting and treatment on patient LEC

| | | | EPO | | | |
|---|---|---|---|---|---|---|
| | | bloodletting | 20000UI/ | fa- | | Blood |
| Name | Dates | 200 ml | sem | tigue | crampes | pressure |
| LEC | 23/04/2008 | 1 | | | | |
| | 30/04/2008 | | | | | |
| | 07/05/2008 | 1 | | | | |
| | 14/05/2008 | | | | | |
| | 29/05/2008 | 1 | | | | |
| | 12/06/2008 | | | | | |
| | 24/06/2008 | 1 | | | * | |
| | 24/07/2008 | 1 | | | | |
| | 14/08/2008 | 1 | | | * | |
| | 12/09/2008 | | 1 | | | |
| | 19/09/2008 | | 1 | | * | |
| | 26/09/2008 | | 1 | | | |
| | 03/10/2008 | | 1 | | | |
| | 10/10/2008 | | 1 | | * | 10/08 |
| | 17/10/2008 | | 1 | | | |
| | 24/10/2008 | | 1 | | | |
| | 31/10/2008 | | 1 | | | |
| | 07/11/2008 | | 1 | | | |
| | 14/11/2008 | | 1 | | | |
| | 21/11/2008 | | 1 | | | |
| | 28/11/2008 | | 1 | | | |
| | 05/12/2008 | | 1 | | | |
| | 10/12/2008 | | 1 | | | 11/6 |
| | 12/12/2008 | | 1 | | | |
| | 19/12/2008 | | 1 | | | |

One injection of EPO 20000UI per week until 24/03/2009

TABLE 1c
Bloodletting and treatment on patient ESP

| | | | EPO | | | |
|---|---|---|---|---|---|---|
| | | bloodletting | 20000UI/ | fa- | | Blood |
| Name | Dates | 200 ml | sem | tigue | crampes | pressure |
| ESP | 23/04/2008 | 1 | | | | |
| | 30/04/2008 | 1 | | | | |
| | 07/05/2008 | 1 | | | | |
| | 14/05/2008 | 1 | | | | |
| | 29/05/2008 | 1 | | | | |
| | 12/06/2008 | 1 | | | | |
| | 24/06/2008 | 1 | | | | |
| | 24/07/2008 | 1 | | | | |
| | 25/08/2008 | 1 | | | | |
| | 12/09/2008 | | | | | |
| | 19/09/2008 | | | | | |
| | 26/09/2008 | | | | | |
| | 03/10/2008 | | 1 | | | 10/6 |
| | 10/10/2008 | | 1 | | | 10/6 |
| | 17/10/2008 | | 1 | | | 10/6 |
| | 24/10/2008 | | 1 | | | 10/6 |
| | 31/10/2008 | | 1 | | | 10/6 |
| | 07/11/2008 | | 1 | | | 10/6 |
| | 14/11/2008 | | 1 | | | 10/6 |
| | 21/11/2008 | | 1 | | | 10/6 |
| | 26/11/2008 | | | | | 11/7 |
| | 28/11/2008 | | 1 | | | |

Ongoing treatment with one injection of EPO 20000UI per week

TABLE 1d
Bloodletting and treatment on patient Til

| | | bloodletting | EPO | | |
|---|---|---|---|---|---|
| Til | Dates | 400 ml | 30000Ul/sem | fatigue | cramps |
| | 25/09/2008 | 1 | | | |
| | 02/10/2008 | 1 | | | |
| | 09/10/2008 | 1 | | | |
| | 16/10/2008 | 1 | | | |
| | 23/10/2008 | 1 | | | |
| | 30/10/2008 | 1 | | | |
| | 06/11/2008 | 1 | | | |
| | 13/11/2008 | 1 | | | |
| | 20/11/2008 | 1 | | | |
| | 27/11/2008 | | | | |
| | 04/12/2008 | 1 | | | |
| | 11/12/2008 | | | | |
| | 18/12/2008 | 1 | | | |
| | 25/12/2008 | | | | |
| | 01/01/2009 | | | | |
| | 08/01/2009 | 1 | | | |
| | 15/01/2009 | | | | |
| | 22/01/2009 | | | | |
| | 29/01/2009 | 1 | | | |
| | 05/02/2009 | | | | |
| | 12/02/2009 | | | | |
| | 19/02/2009 | 1 | | * | |
| | 26/02/2009 | | | | |
| | 05/03/2009 | | | | |
| | 12/03/2009 | 1 | | * | |
| | 19/03/2009 | | | | |
| | 26/03/2009 | | | | |
| | 02/04/2009 | | | | |
| | 09/04/2009 | 1 | | * | |
| | 16/04/2009 | | | | |
| | 23/04/2009 | | | | |
| | 30/04/2009 | | | | |
| | 07/05/2009 | | | | |
| | 14/05/2009 | | 1 | | |
| | 21/05/2009 | | 1 | | |
| | 28/05/2009 | | 1 | | |
| | 04/06/2009 | | 1 | | |
| | 11/06/2009 | | 1 | | |
| | 18/06/2009 | | 1 | | |
| | 25/06/2009 | | 1 | | |
| | 02/07/2009 | | 1 | | |
| | 09/07/2009 | | 1 | | |

| | one injection each week | |
|---|---|---|
| Table 1d | 11/01/2010 | 1 |

Treatment of patient Til is ongoing with one injection of EPO 30000 UI per week.

TABLE 1E
the treatment phases

| | Bloodletting | Bloodletting | EPO | EPO |
|---|---|---|---|---|
| Patient | initiation | interruption | initiation | interruption |
| PER | 23/04/2008 | 25/08/2008 | 12/09/2008 | 15/12/2008 |
| LEC | 23/04/2008 | 14/08/2008 | 12/09/2008 | 24/03/2009 |
| ESP | 23/04/2008 | 25/08/2008 | 03/10/2008 | Ongoing |
| TIL | 25/09/2008 | 09/04/2009 | 14/05/2009 | Ongoing |

Methods

Clinical Assessment

Each patient underwent the following clinical scales: Expanded Disability Status Scale (EDSS) (Kurtzke, 1983), time to walk 10 meters, maximum walking distance, MSWS12 functional scale (Hobart et al., 2003), fatigue severity scale (Krupp et al., 1989). Evaluations were performed before bloodletting, 3 months after first bloodletting and at 6 months.

Neurophysiological Assessment

Motor cortex excitability testing: Subjects were seated in a comfortable reclining chair with a tightly fitting Lycra swimming cap placed over the head. They were instructed to keep their hands as relaxed as possible. Transcranial magnetic stimulation (TMS) was performed with a Magstim 200 stimulator (Magstim Company, Carmarthenshire, UK) and a figure-of-eight double 70-mm coil (no. 9925-00, Magstim). Two Magstim 200 stimulators connected through a Bistim module served to deliver paired pulses. The optimal site for evoking motor responses in the first dorsalis interosseus (FDI) muscles was determined over the scalp (motor hot spot) and marked on the cap. The motor evoked potentials (MEPs) were recorded through a 20- to 1,000-Hz bandpass using a standard electromyograph (Phasis II, EsaOte, Florence, Italy) and pre-gelled self-adhesive disposable surface electrodes (no. 9013S0241, Medtronic Functional Diagnostics, Skovlunde, Denmark), placed on the belly and tendon of the FDI muscle. A Velcro bracelet was strapped around the forearm as ground electrode (no. 9013S0711, Medtronic). The coil was positioned tangentially to the surface of the head, with the handle pointing occipitally along a sagittal axis.

1. REST MOTOR THRESHOLD (RMT) was defined as the minimal intensity of stimulation required to elicit MEPs of >50 μV in amplitude in at least five of 10 trials performed during complete muscle relaxation.

2. The relationship between stimulus intensity and MEP amplitude was assessed by studying the most variable part of the stimulus/response curve. This was previously found to correspond to TMS intensities ranging between 120 and 140% of RMT. The amplitude ratio of the MEP obtained at 140% of RMT to that obtained at 120% of RMT (140/120 r) was therefore calculated.

3. The CORTICOSPINAL SILENT PERIOD (CSP) was determined as the duration of the post-MEP EMG activity interruption following single TMS pulses delivered at 140% of RMT. Stimulations were performed while patients exerted a tonic maximal voluntary contraction of the FDI muscle against the examiner's resistance. Four rectified traces, each consisting of three averaged trials, were superimposed. The minimal CSP duration was measured from the end of the MEP until the first reoccurrence of EMG activity.

4. Finally, paired-pulse paradigms were applied, with a conditioning stimulus set at 80% of RMT and a test stimulus set at 120% of RMT, while the FDI muscle was at rest. Various interstimuli intervals (ISIs) were randomized (2 and 4 msec for INTRACORTICAL INHIBITION (ICI); 10 and 15 msec for INTRACORTICAL FACILITATION (ICF)) and intermixed with control trials (test stimulus alone). For each ISI, four trials were averaged and the resulting MEP amplitude was converted into a percentage of the control MEP amplitude (pp/cMEP %). Paired-pulse parameters were expressed as the amount of inhibition (ICI_{—}100%_pp/cMEP %) and facilitation (ICF pp/cMEP %_{—}100%). The maximum degrees of inhibition and facilitation achieved at any ISI were retained for analysis.

5. CEREBELLOTHALAMOCORTICAL (CTC) PATHWAYS. Stimulation of the left cerebellum was performed with a double-cone coil centered 3-4 cm lateral to the inion. Stimulus intensity was set at 5-10% below the active motor threshold. Stimulation of the left hand representation in the right M1 was performed with a figure-of-eight coil oriented at 45° to the midsagittal line. Stimulus intensity was adjusted to elicit motor evoked potentials (MEPs) with peak-to-peak amplitude of 0.5-1 mV in the relaxed first dorsal interosseous (FDI) muscle. Interstimulus intervals between cerebellar and M1 stimulations ranged from 5 to 8 ms. The amplitude of the FDI-MEPs to M1 stimulation was compared between conditioned and unconditioned trials to assess the effect of CTC pathway activation by cerebellar stimulation.

INTERHEMISPHERIC INHIBITION. Focal TMS of the motor cortex was performed with a figure-of-eight coil (outside diameter of half-coil, 8.5 cm) of the Magstim 200 stimulator (2-Tesla version; Magstim Co., Dyfed, UK) to elicit corticospinally mediated contralateral electromyographic (EMG) responses in the first dorsal interosseous muscle (FDI) and TRANSCALLOSAL INHIBITION (TI) of tonic voluntary EMG activity in the FDI ipsilateral to stimulation. TMS was performed during maximal tonic muscle contraction over the individually determined point from which maximal EMG responses could be obtained. In the axis of the stimulation coil, the currents were directed anteroposteriorly (induced currents with opposite orientation) because this direction is the most effective for eliciting TI. To prevent fatigue the patients were asked to relax their hand muscles for about 3 seconds after each stimulus. Cortical stimulation was performed with 80% of the maximum stimulator output. For such a stimulus intensity, TI could always be elicited in normal subjects, and the onset latency and duration of TI did not further change with increasing stimulus intensities. Twenty consecutive EMG traces were recorded. Onset latency and duration of TI were determined for rectified and averaged EMG activity of the FDI ipsilateral to cortex stimulation. The onset latency of TI was measured from the stimulus to the point where the signal of the averaged tonic EMG activity clearly fell under the mean amplitude of the EMG activity before the stimulus. The duration of TI was measured from the onset of TI to a point where the EMG activity reached the mean amplitude of the baseline EMG activity before the stimulus. The transcallosal conduction time was determined by subtracting the onset latency of the corticospinally mediated EMG response from the onset latency of TI in the same FDI.

Biological Assessment

Erythrocytes count, haemoglobin concentration, mean corpuscular volume (MCV), red cells distribution width (RDW), iron concentration, ferritin concentration, transferrrin and transferrrin saturation, erythrocyte ferritin, circulating erythropoietin were measured before each bloodletting.

Bloodletting

All four patients underwent the same process that consisted of depletion of iron content through the performance of successive bloodlettings (200 to 250 ml for each bloodletting) that were performed each week for the first six weeks, then according to biological changes and clinical tolerance.

Blood cell counts and iron content were systematically evaluated before and during the performance of bloodletting. The objective was the reduction of a multibillion and to 11 g per litre. The tolerance and the safety of the process have been assessed (tables 1a,b,c,d).

Treatment

Erythropoietin: after reaching a level of 11 g per litre of haemoglobin and low ferritin level (below 50 μg/l), supporting the iron depletion, patients were invited to begin the treatment with erythropoietin. The weekly dose of rhEPO has been set at 300 UI/kg (20000 UI/week) once a week in the 3 patients for 24 weeks in addition to the monitoring of blood parameters (tables 1a, 1b, 1c and 1d).

Results

Clinical Results

Bloodlettings were well tolerated. However, some fatigue, especially during physical effort, was observed when the patients reached an haemoglobin concentration of 11 g/l. No patient experienced ischemic manifestations. Two of them experienced muscle cramps in the legs. Clinical scales are resumed in tables 2a, 2b, 2c and 2d.

Erythropoietin was well tolerated. One patient experienced mild headache the day after injection. She also had 2 broken teeth (teeth with previous traumatisms).

Muscle cramps disappeared after initiation of rhEPO.

Two of the four patients had improved fatigue scale (FSS) after 3 months of EPO, one patient had improved time for 10 meters walk. This patient had also improved maximum walking distance after initial decline before EPO initiation, decreased fatigability for speaking, reading, driving.

The treatment was prolonged in patient Esp; the effect of the erythropoietin is prolonged after one year as attested by the clinical and neurophysiological persistent effect in patient three (Esp); it is noteworthy that the iron depletion is still present nine months after treatment by erythropoietin (see patient Esp) who did not required new bloodlettings.

The patient Lec interrupted voluntarily the EPO treatment. The results clearly show a loss of effect with a decrease of motor amplitude (120%) after interruption (see table 4b and table 1E). A similar effect is observed in patient Per who interrupted treatment after 4 months.

TABLE 2a
Clinical results (patient PER)

| | Name | | | | | |
|---|---|---|---|---|---|---|
| | PER | | | | | |
| | Dates | | | | | |
| | 12/06/2008 | 09/07/2008 | 10/09/2008 | 15/10/2008 | 10/12/2008 | |
| Clinical evaluation | before bloodletting | after bloodletting | before rhEPO | After rhEPO | After rhEPO | |
| MSWS12 | 47 | 49 | 50 | 55 | 55 | pt |
| Max walking capacity | 50 | 57 | 12 | 36 | 2 | m |
| 10 m walk | NA | NA | NA | NA | NA | |
| EDSS | 6.5 | 6.5 | 6.5 | 6.5 | 7 | |
| FSS | 55 | 58 | 56 | 63 | 60 | pt |

TABLE 2b
Clinical results (patient LEC)

| | Name | | | | |
|---|---|---|---|---|---|
| | LEC | | | | |
| | Dates | | | | |
| | | | 15/10/ | 10/12/ | |
| | 12/06/2008 | 10/09/2008 | 2008 | 2008 | |
| Clinical | before | before | after | after | |
| evaluation | bloodletting | EPO | rhEPO | rhEPO | |
| MSWS12 | 51 | 55 | 57 | 57 | pt |
| Max walking | 850 | 460 | 200 | 330 | m |
| capacity | | | | | |
| 10 m walk | 13.5 | 13.5 | 13 | 12.5 | |
| EDSS | 5 | 5 | 5 | 5 | |
| FSS | 54 | 58 | 59 | 34 | pt |
| Miscellaneous | | | | can read, | |
| | | | | talk, drive | |

TABLE 2c
Clinical results (patient ESP)

| | Name | | | | | | | |
|---|---|---|---|---|---|---|---|---|
| | ESP | | | TABLE 2c suppl | | | | |
| | Dates | | | | | | | |
| | 17/04/2008 | | | | | | | |
| Clinical | before | 09/07/2008 | 10/09/2008 | 26/11/2008 | 11/02/2009 | 21/10/209 | 13/01/2010 | |
| evaluation | bloodletting | after bloodletting | before rhEPO | after rhEPO | after rhEPO | after rhEPO | after rhEPO | |
| MSWS12 | 48 | 40 | | | | | | pt |
| Max walking | 2 | 2 | 3 | 2 | 2 | 2 | 2 | m |
| capacity | | | | | | | | |
| 10 m walk | impossible | impossible | impossible | impossible | impossible | impossible | impossible | |
| EDSS | 7 | | | 7 | 7 | 7 | 7 | |
| FSS | 49 | 36 | | 34 | 36 | 34 | 35 | pt |
| Miscellaneous | | | | can cough, | can cough, | can cough, talk | can cough, talk | |
| | | | | perform | perform | louder | louder | |
| | | | | transfer, talk | transfer, talk | | | |
| | | | | louder | louder | | | |

TABLE 2d
Clinical results (patient Til)

| | Name | | | | | | |
|---|---|---|---|---|---|---|---|
| | Til | | | | | Table 2d | |
| | Dates | | | | | | |
| | | 07/01/2009 | 13/03/2009 | 03/06/2009 | 21/10/2009 | 13/01/2010 | |
| | 24/09/2008 | after | before | 1 M after | 5 M after | 8 M after | |
| Clinical evaluation | before bloodletting | bloodletting | rhEPO | rhEPO | rhEPO | hEPO | |
| MSWS12 | 51 | 48 | 46 | 49 | 52 | 32 | pt |
| Max walking | 70 | 60 | 50 | 115 | 120 | 150 | m |
| capacity | | | | | | | |
| 10 m walk | 22 | 20 | 19 | 17 | 17 | 17 | |
| EDSS | 6.5 | 6 | 6.5 | 6.5 | 6.5 | 6.5 | |
| FSS | 59 | 44 | 49 | 41 | 41 | 41 | pt |

Biological Results.

The biological results are synthesized in tables 3 a,b,c,d.

The tolerance of EPO was excellent without rise of haemoglobin concentration due to iron deficiency.

TABLE 3a
Biological results (patient PER)

| | | | | | | | serum | | Transferrin | Erythrocyte | |
|---|---|---|---|---|---|---|---|---|---|---|---|
| Name | Dates | Erythrocytes | Haemoglobin | MCV | RDW | Iron | ferritin | Transferrin | saturation | ferritin | EPO |
| PER | 17/04/2008 | 3.8 | 12.1 | 96 | 13 | 18.8 | 191 | 3.58 | 21 | | 6.4 |
| | 23/04/2008 | 3.6 | 11.4 | 96 | 14 | 9.6 | 186 | 3.48 | 11 | | |
| | 30/04/2008 | 3.6 | 11.5 | 97 | 14 | 15.5 | 145 | 3.89 | 16 | | |
| | 14/05/2008 | 3.6 | 11.2 | 96 | 14 | 14.3 | 94 | 3.74 | 15 | 25.2 | |
| | 29/05/2008 | 3.6 | 11.3 | 96 | 14 | 18.3 | 62 | 3.71 | 20 | 15.5 | 12.81 |
| | 12/06/2008 | 3.5 | 11.1 | 98 | 14 | 15.5 | 35 | 3.54 | 18 | 14.1 | 12.81 |
| | 24/06/2008 | 3.8 | 11.7 | 95 | 14 | 13 | 33 | 3.6 | 14 | 17.1 | 12.11 |
| | 24/07/2008 | 3.5 | 11.2 | 95 | 14 | 10.7 | 15 | 3.79 | 11 | 14.4 | 11.6 |
| | 25/08/2008 | 3.7 | 11.3 | 91 | 14 | 9.1 | 17 | 4.30 | 8 | 5.7 | 15.3 |
| After | 15/10/2008 | 4.2 | 11.2 | 84 | 16 | 3.5 | 14 | 4.36 | 3 | | |
| rhEPO | | | | | | | | | | | |
| After | 11/12/2008 | 4.6 | 11.1 | 78 | 19 | 3.8 | 28 | 3.63 | 4 | | |
| rhEPO | | | | | | | | | | | |

TABLE 3b
Biological results (patient LEC)

| | | | | | | | serum | | Transferrin | Erythrocyte | |
|---|---|---|---|---|---|---|---|---|---|---|---|
| Name | Dates | Erythrocytes | Haemoglobin | MCV | RDW | Iron | ferritin | Transferrin | saturation | ferritin | EPO |
| LEC | 01/04/2008 | 4.8 | 15.1 | 94 | 14 | | | | | | |
| | 12/06/2008 | 5 | 15.6 | 95 | 14 | 12.9 | 57 | 2.39 | 60 | 40.3 | 4.37 |
| | 19/06/2008 | 4.4 | 13.5 | 93 | 14 | 12.4 | 34 | 2.27 | 57 | 54.2 | 19.22 |
| | 26/06/2008 | 4 | 12.5 | 94 | 14 | | 26 | | | 52.8 | 24.53 |
| | 04/07/2008 | 3.9 | 12 | 92 | 14 | 4.7 | 18 | 2.46 | 62 | 47.6 | 38.59 |
| | 24/07/2008 | 3.8 | 11.1 | 89 | 14 | | 15 | | | 34.6 | 48.44 |
| | 14/08/2008 | 4.1 | 11.4 | 84 | 16 | 4 | 12 | 3.24 | 81 | | |
| after | 15/10/2008 | 4.7 | 10.7 | 71 | 20 | 2.5 | 10 | 3.36 | 3 | 4.9 | |
| rhEPO | | | | | | | | | | | |
| after | 10/12/2008 | 5.4 | 11.6 | 69 | 22 | 3.1 | 7 | 3.36 | 4 | | |
| rhEPO | | | | | | | | | | | |

TABLE 3c
Biological results (patient ESP)

| | | | | | | | serum | | Transferrin | Erythrocyte | |
|---|---|---|---|---|---|---|---|---|---|---|---|
| Name | Dates | Erythrocytes | Haemoglobin | MCV | RDW | Iron | ferritin | Transferrin | saturation | ferritin | EPO |
| ESP | 17/04/2008 | 4 | 12.4 | 92 | 14 | 17.8 | 108 | 2.46 | 29 | 105 | 4.68 |
| | 07/05/2008 | 3.5 | 10.9 | 95 | 16 | 12.6 | 29 | 2.51 | 20 | 118.2 | 24.06 |
| | 29/05/2008 | | | | | 18 | 24 | 2.58 | 28 | 77.4 | |
| | 19/06/2008 | 3.9 | 11.9 | 92 | 14 | 9.3 | 9 | 2.76 | 13 | | 16.25 |
| | 09/07/2008 | 3.7 | 11.3 | 90 | 13 | 11.2 | 7 | 2.69 | 17 | 49 | 14.22 |
| | 31/07/2008 | 3.8 | 11.6 | 91 | 14 | 7.3 | 7 | 3.33 | 9 | 35.9 | 17.3 |
| | 28/08/2008 | 3.6 | 10.5 | 89 | 14 | 7.2 | 6 | 3.02 | 10 | 17.2 | |
| after | 26/11/2008 | 4.7 | 11.3 | 77 | 19 | 2.7 | 3 | 3.2 | 3 | 8 | |
| rhEPO | | | | | | | | | | | |

TABLE 3d
Biological results (patient Til)

| | Name | | | | | |
|---|---|---|---|---|---|---|
| | Til | | | | Table 3d | |
| | Dates | | | | | |
| | | | | | | 01/04/2009 |
| Biological follow-up | 24/09/2008 | 01/10/2009 | 22/10/2008 | 19/11/2008 | 07/01/2009 | before rhEPO |
| Erythrocytes | 4.6 | 4.1 | | 4.2 | | 4.1 |
| Haemoglobin | 14.3 | 13.1 | | 13.1 | | 11.6 |
| MCV | 91 | 92 | | 93 | | 87 |
| RDW | 14 | 13 | | 14 | | 14 |
| Iron | 9 | | 13 | | 8.2 | 4.9 |
| serum ferritin | 818 | | 386 | | 59 | * 11 |
| Transferrin | 2.09 | | 2.28 | | 3.04 | 3.26⁽³⁾ |
| Transferrin saturation | 17 | | 23 | | 11 | 6 |
| Erythrocyte ferritin | | | 17.1 | | 10.7 | |
| endogenous EPO | 6.71 | | 15.8 | 8.5 | 19 | |

Neurophysiological Results.

All four patients had a similar neurophysiological change with improved cortex excitability that persisted after 24 weeks. Changes were characterised by improved active motor threshold, amplitude (120%) of motor potential, intracortical facilitation and decreased long intracortical inhibition and interhemispheric inhibition (two patients).

Neurophysiological changes were observed after the first step with iron depletion and persisted or improved after initiation of rhEPO.

All the results are summarised in tables 4 a, 4b.1, 4b.2, 4c and 4d.

Results are synthesized in tables 5 and 6.

TABLE 4a
Neurophysiological results (patient PER)

| | Name | | | | | |
|---|---|---|---|---|---|---|
| | PER | TABLE 4a suppl | | | | |
| | Dates | | | | | |
| | 16/04/2008 | 10/07/2008 | | 15/10/2008 | | 08/04/2009 |
| | before | after | 10/09/2008 | After | 11/12/2008 | POST-EPO |
| CORTICAL EXCITABILITY | bloodletting | bloodletting | before rhEPO | rhEPO 1 M | POST-EPO 3 M | 7 M |
| REST MOTOR THRESHOLD | 70% | 74% | 68% | 68% | 65% | 52% |
| ACTIVE MOTOR THRESHOLD | 75% | 74% | 72% | 65% | 55% | 48% |
| LATENCY (120%): | 26 ms | 26 ms | 22 ms | 22 ms | 21 ms | 26 ms |
| AMPLITUDE (120%): | 68 μV | 44 μV | 160 μV | 213 μV | 198 μV | 49 μV |
| RATIO 140/120: | 2.56 | 1.45 | 1.48 | 1.35 | 1.63 | 2.16 |
| SHORT INTRACORTICAL INHIBITION | −72% | −76% | −18% | −52% | −4% | −68% |
| SHORT INTRACORTICAL INHIBITION (mean 2-4 ms) | −18% | −75% | −3% | −35% | −4% | −62% |
| INTRACORTICAL FACILITATION | +14% | +403% | +88% | +113% | +264% | +12% |
| INTRACORTICAL FACILITATION (mean 10-15 ms) | +11% | +397% | +69% | +98% | +122% | +11% |
| LONG INTRACORTICAL INHIBITION (100 ms): | −100% | −100% | −100% | −100% | −59% | −100% |
| LONG INTRACORTICAL INHIBITION (150 ms): | −100% | −100% | −28% | −100% | −82% | −100% |
| DURATION cSP (120%) (circle) | 203 ms | 198 ms | 143 ms | 227 ms | 120 ms | 111 ms |
| DURATION cSP post-MEP (120%): | 162 ms | 137 ms | 96 ms | 134 ms | 66 ms | 54 ms |
| RATIO cSP/MEP (120%): | 1.47 | 1.17 | 0.44 | 0.40 | 0.12 | 0.06 |
| RATIO 140/120: | 1.48 | 1.52 | 1.50 | 1.19 | 1.07 | 1.06 |
| DURATION cSP (figure of 8) | 179 ms | 50 ms | 64 ms | 158 ms | 62 ms | abs ms |
| LATENCY iSP: | 47 ms | abs ms | 47 ms | abs ms | abs ms | abs ms |
| DURATION iSP: | 24 ms | abs ms | 55 ms | abs ms | abs ms | abs ms |
| TCTC: | 21 ms | abs ms | 25 ms | abs ms | abs ms | abs ms |
| INTERHEMISPHERIC INHIBITION (max) | −33% | −4% | −65% | −26% | −85% | −67% |
| INTERHEMISPHERIC INHIBITION (mean 8-10-12) | −17% | +16% | −59% | −13% | −73% | −39% |
| CEREBELLOTHALAMOCORTICAL INHIBITION 5 | +16% | −6% | −21% | −56% | −40% | −15% |
| max | | | | | | |
| CEREBELLOTHALAMOCORTICAL INHIBITION moy: | +21% | +8% | −14% | −39% | −25% | −3% |

“POST-EPO X M” means that measures are made X months following the initiation of the EPO treatment.

Last data observed for patient Per (on Aug. 4, 2009 “POST-EPO 7M”) followed an interruption of EPO treatment of 4 months.

TABLE 4b.1
Neurophysiological results (patient LEC)

| | Name | | | |
|---|---|---|---|---|
| | LEC | | | |
| | Dates | | | |
| | 12/06/2008 | 10/09/2008 | 15/10/2008 | 11/12/2008 |
| | before | after | after | after |
| CORTICAL EXCITABILITY | bloodletting | bloodletting | rhEPO 1M | rhEPO 3M |
| REST MOTOR THRESHOLD | imp % | imp % | 95% | 85% |
| ACTIVE MOTOR THRESHOLD | 100% | 100% | 90% | 70% |
| LATENCY (120%): | 23 (actif) ms | 25 (actif) ms | 23 ms | 26 ms |
| AMPLITUDE (120%): | 228.7 (actif) μV | 143.1 (actif) μV | 267.3 μV | 403.8 μV |
| RATIO 140/120: | 1.29 (actif) | 2.11 (actif) | imp | imp |
| SHORT INTRACORTICAL INHIBITION | imp % | imp % | nf % | −32.4% |
| INTRACORTICAL FACILITATION | imp % | imp % | nf % | +370.2% |
| LONG INTRACORTICAL INHIBITION | nf % | +34.6% | +26.7% | +589.4% |
| (100 ms): | | | | |
| LONG INTRACORTICAL INHIBITION | nf % | +80.7% | −27.7% | +19.9% |
| (150 ms): | | | | |
| DURATION cSP (120%) (circle) | nf ms | nf ms | 95.2 ms | 95.6 ms |
| DURATION cSP post-MEP (120%): | nf ms | nf ms | 43.6 ms | 48.8 ms |
| RATIO cSP/MEP (120%): | nf | nf | 0.05 | 0.1 |
| RATIO 140/120: | nf | nf | imp | imp |
| DURATION cSP (figure of 8) | abs ms | 39 ms | 41.6 ms | 52.4 ms |
| LATENCY iSP: | abs ms | abs ms | abs ms | abs ms |
| DURATION iSP: | abs ms | abs ms | abs ms | abs ms |
| TCTC: | abs ms | abs ms | abs ms | abs ms |
| INTERHEMISPHERIC INHIBITION | imp % | nf % | −10.9% | −29.6% |
| CEREBELLOTHALAMOCORTICAL | imp % | −57.8% | −73.6% | −60.1% |
| INHIBITION 5. max: | | | | |
| CEREBELLOTHALAMOCORTICAL | imp % | −33.6% | −52.1% | −47.4% |
| INHIBITION moy: | | | | |

TABLE 4b.2
Neurophysiological results (patient LEC)

| | Dates | |
|---|---|---|
| | | 23/06/2009 |
| | | Post-EPO 9M |
| | | (following |
| | | 3 months |
| | 25/03/2009 | of interruption |
| | Post-EPO | of the |
| CORTICAL EXCITABILITY | 6M | EPO treatment) |
| REST MOTOR THRESHOLD | 78% | 82% |
| ACTIVE MOTOR THRESHOLD | 67% | 88% |
| LATENCY (120%): | 26 ms | 26 ms |
| AMPLITUDE (120%): | 244 μV | 100 μV |
| RATIO 140/120: | imp | imp |
| SHORT INTRACORTICAL | −37% | −85% |
| INHIBITION | | |
| SHORT INTRACORTICAL | −7% | −71% |
| INHIBITION (mean 2-4 ms) | | |
| INTRACORTICAL FACILITATION | −8% | +196% |
| INTRACORTICAL FACILITATION | −23% | +27% |
| (mean 10-15 ms) | | |
| LONG INTRACORTICAL | +21% | +227% |
| INHIBITION (100 ms): | | |
| LONG INTRACORTICAL | −39% | +184% |
| INHIBITION (150 ms): | | |
| DURATION cSP (120%) (circle) | 112 ms | 105 ms |
| DURATION cSP post-MEP (120%) | 64 ms | 54 ms |
| RATIO cSP/MEP (120%): | 0.12 | 0.15 |
| RATIO 140/120: | imp | imp |
| DURATION cSP (figure of 8) | Abs ms | Abs ms |
| LATENCY iSP: | Abs ms | Abs ms |
| DURATION iSP: | Abs ms | Abs ms |
| TCTC: | Abs ms | Abs ms |
| INTERHEMISPHERIC INHIBITION | −41% | Abs (PEM f-8) % |
| (max) | | |
| INTERHEMISPHERIC INHIBITION | −14% | Abs |
| (mean 8-10-12) | | |
| CEREBELLOTHALAMOCORTICAL | −13% | Abs % |
| INHIBITION 5. max: | | |
| CEREBELLOTHALAMOCORTICAL | −2% | Abs % |
| INHIBITION moy: | | |

TABLE 4c
Neurophysiological results (patient ESP)

| Name | |
|---|---|
| ESP | TABLE 4c. suppl |

Dates

| | 16/04/2008 | 10/07/2008 | 10/09/2008 | 27/11/2008 | 21/04/2009 | 09/12/2009 |
|---|---|---|---|---|---|---|
| | before | after | before | After | POST-EPO | POST-EPO |
| CORTICAL EXCITABILITY | bloodlett | bloodletting | rhEPO | rhEPO 1 M | 6 M | 12 M |
| REST MOTOR THRESHOLD | 100% | 100% | 100% | 100% | 97% | 100% |
| ACTIVE MOTOR THRESHOLD | 100% | 75% | 65% | 75% | 77% | 95% |
| LATENCY (120%): | 18 ms | 21 ms | 20 ms | 19 ms | 22 ms | 22 ms |
| AMPLITUDE (120%): | 182.6 μV | 128.3 μV | 88.1 μV | 232.1 μV | 460 μV | 136 μV |
| RATIO 140/120: | imp | imp | imp | imp | imp | imp |
| SHORT INTRACORTICAL INHIBITION | −13% | −41% | −77% | −59% | −65% | −30% |
| SHORT INTRACORTICAL INHIBITION (mean 2-4 ms) | −11% | −15% | −51% | −58% | −52% | −24% |
| INTRACORTICAL FACILITATION | +28% | +444% | +293% | +216% | +173% | +43% |
| INTRACORTICAL FACILITATION (mean 10-15 ms) | +23% | +305% | +114% | +40% | +121% | +40% |
| LONG INTRACORTICAL INHIBITION (100 ms): | −28.7% | −12.7% | +102.8% | +137% | −19% | −43% |
| LONG INTRACORTICAL INHIBITION (150 ms): | −52.4% | −18% | +54.6% | +53.3% | +62% | −62% |
| DURATION cSP (120%) (circle) | 89 ms | 153 ms | 181 ms | 137 ms | 127 ms | 117 ms |
| DURATION cSP post-MEP (120%): | 48 ms | 109 ms | 121 ms | 92 ms | 85 ms | 71 ms |
| RATIO cSP/MEP (120%): | 0.27 | 0.4 | 0.5 | 0.36 | 0.24 | 0.50 |
| RATIO 140/120: | 1.31 | 1.48 | 1.49 | 1.24 | nd | nd |
| DURATION cSP (figure of 8) | abs ms | 48 ms | 25 ms | 35 ms | abs ms | abs ms |
| LATENCY iSP: | abs ms | abs ms | abs ms | abs ms | abs ms | abs ms |
| DURATION iSP: | abs ms | abs ms | abs ms | abs ms | abs ms | abs ms |
| TCTC: | abs ms | abs ms | abs ms | abs ms | abs ms | abs ms |
| INTERHEMISPHERIC INHIBITION (max) | −73.7% | −79.5% | −63.8% | −60.5% | −43% | abs (PEM f-8) % |
| INTERHEMISPHERIC INHIBITION (mean 8-10-12) | −43% | −59% | −51% | −35% | −20% | abs % |
| CEREBELLOTHALAMOCORTICAL INHIBITION 5. | −37.8% | −21.6% | −50.5% | −14.7% | −45% | abs % |
| max | | | | | | |
| CEREBELLOTHALAMOCORTICAL INHIBITION moy: | −9.2% | −6.2% | −29.8% | −5.5% | −34% | abs % |

TABLE 4d
Neurophysiological results (patient Til)

| | Name | | | | |
|---|---|---|---|---|---|
| | TIL Table 4d | | | | |
| | Dates | | | | |
| | 12/06/2008 | 08/01/2009 | 13/02/2009 | 02/06/2009 | 09/12/2009 |
| | before | after | before | POST-EPO | POST-EPO |
| CORTICAL EXCITABILITY | bloodletting | bloodletting | rhEPO | 1M | 7M |
| REST MOTOR THRESHOLD | 75% | 68% | 65% | 60% | 48% |
| ACTIVE MOTOR THRESHOLD | 82% | 62% | 52% | 45% | 40% |
| LATENCY (120%): | 27 ms | 27 ms | 24 ms | 26 ms | 24 ms |
| AMPLITUDE (120%): | 128.2 μV | 105.1 μV | 184.2 μV | 96 μV | 494 μV |
| RATIO 140/120: | 2.04 | 1.76 | 2.29 | 1.88 | 2.22 |
| SHORT INTRACORTICAL INHIBITION | −38.9% | −15.9% | −76.4% | −5% | −57% |
| SHORT INTRACORTICAL INHIBITION (mean 2-4 ms) | | | | +3% | −55% |
| INTRACORTICAL FACILITATION | +75.5% | +502.2% | +285.1% | +346% | +95% |
| INTRACORTICAL FACILITATION (mean 2-4 ms) | | | | +278% | +86% |
| LONG INTRACORTICAL INHIBITION (100 ms): | −80.5% | −51.5% | 0% | −85% | −27% |
| LONG INTRACORTICAL INHIBITION (150 ms): | −72% | −41.1% | 0% | −80% | +4% |
| DURATION cSP (120%) (circle) | 292 ms | 331 ms | 243 ms | 275 ms | 290 ms |
| DURATION cSP post-MEP (120%): | 219 ms | 263 ms | 178 ms | 213 ms | 232 ms |
| RATIO cSP/MEP (120%): | 0.4 | 0.42 | 0.55 | 0.78 | 0.50 |
| RATIO 140/120: | 1.94 | 1.13 | 1.22 | 1.52 | 1.42 |
| DURATION cSP (figure of 8) | 113 ms | 202 ms | nf ms | nf ms | nf ms |
| LATENCY iSP: | abs ms | 53 ms | nf ms | nf ms | nf ms |
| DURATION iSP: | abs ms | 57 ms | nf ms | nf ms | nf ms |
| TCTC: | abs ms | 26 ms | nf ms | nf ms | nf ms |
| INTERHEMISPHERIC INHIBITION (max) | −54% | −63% | −64% | −65% | −63% |
| INTERHEMISPHERIC INHIBITION (mean 8-10-12) | −19% | −21% | −46% | −49% | −48% |
| CEREBELLOTHALAMOCORTICAL INHIBITION 5. | −25% | −45% | −52% | −47% | −48% |
| CEREBELLOTHALAMOCORTICAL INHIBITION moy: | +2% | −15% | −31% | −27% | −22% |

TABLE 5
Results synthesis

| | decreased | unchanged | increased |
|---|---|---|---|
| CLINICAL SCALES | | | |
| MSWS12* | 1 | 1 | 2 |
| Expanded Disability Status Scale | 0 | 3 | 1 |
| (EDSS)* | | | |
| Time to walk 10 meters* | 2 | 2 | 0 |
| Maximum walking distance | 2 | 2 | 2 |
| Fatigue severity scale* | 3 | 0 | 1 |
| Miscellaneous | 3 | 1 | 3 |
| BIOLOGICAL PARAMETERS | | | |
| Iron | 4 | 0 | 0 |
| Natural EPO | 0 | 0 | 4 |
| Erythropoiesis rhEPO related | 0 | 0 | 4 |
| CORTICAL EXCITABILITY | | | |
| REST MOTOR THRESHOLD | 2 | 2 | 0 |
| ACTIVE MOTOR THRESHOLD | 4 | 0 | 0 |
| LATENCY (120%): | 1 | 3 | 0 |
| AMPLITUDE (120%): | 0 | 0 | 4 |
| RATIO 140/120: | 1 | 1 | 0 |
| SHORT INTRACORTICAL | 2 | 0 | 1 |
| INHIBITION | | | |
| INTRACORTICAL FACILITATION | 0 | 0 | 4 |
| LONG INTRACORTICAL | 4 | 0 | 0 |
| INHIBITION (100 ms): | | | |
| LONG INTRACORTICAL | 3 | 0 | 1 |
| INHIBITION (150 ms): | | | |
| DURATION cSP (120%) (circle) | 1 | 1 | 1 |
| DURATION cSP post-MEP (120%): | 1 | 1 | 1 |
| RATIO cSP/MEP (120%): | 1 | 2 | 0 |
| RATIO 140/120: | 2 | 1 | 0 |
| DURATION cSP (figure of 8) | 1 | 2 | 0 |
| LATENCY iSP: | 0 | 3 | 0 |
| DURATION iSP: | 0 | 3 | 0 |
| TCTC: | 0 | 3 | 0 |
| INTERHEMISPHERIC INHIBITION | 1 | 1 | 2 |
| CEREBELLOTHALAMO- | 1 | 2 | 1 |
| CORTICAL INHIBITION 5. max: | | | |
| CEREBELLOTHALAMO- | 1 | 2 | 1 |
| CORTICAL INHIBITION moy: | | | |

*better if decreased

TABLE 6
Global synthesis

| | | | | | Improvement |
|---|---|---|---|---|---|
| PARAMETERS | Per | Esp | Lec | Til | (n =) |
| CLINICAL | | | | | |
| MSWS12* | + | = | + | − | 1 |
| Expanded Disability Status | + | = | = | − | 0 |
| Scale (EDSS)* | | | | | |
| Time to walk 10 meters* | = | = | − | − | 2 |
| Maximum walking distance | − | = | + | + | 2 |
| Fatigue severity scale* | + | − | − | − | 3 |
| Miscellaneous | = | + | + | + | 3 |
| BIOLOGICAL | | | | | |
| Iron (decreased ratio) | 2.1 | 2.5 | 3.2 | 1.8 | 4 |
| Natural EPO (increased ratio) | 2.4 | 3.7 | 11.1 | 2.8 | 4 |
| Ezrythtropoiesis rhEPO related (%) | 33 | 30 | 31 | 26 | 4 |
| NEUROPHYSIOLOGICAL | | | | | |
| ACTIVE MOTOR THRESHOLD | + | + | + | + | 4 |
| AMPLITUDE (120%): | + | + | + | + | 4 |
| INTRACORTICAL | + | + | + | + | 4 |
| FACILITATION | | | | | |
| LONG INTRACORTICAL | − | − | − | − | 4 |
| INHIBITION (100 ms): | | | | | |
| INTERHEMISPHERIC | − | = | − | + | 2 |
| INHIBITION | | | | | |

Discussion

In the present study, the inventors have shown that bloodletting with iron-induced deficiency was associated with an improvement of intra-cortical facilitation and motor threshold in the central nervous system. They have also shown that rhEPO initiated after iron deficiency increased theses changes. The intra-cortical facilitation is related to glutamate and glutamate receptor physiology. The multiple sclerosis glutamate physiology is altered in multiple sclerosis as a consequence of local inflammatory changes. The latter induce depolarization and an influx of Na+, Ca overload therefore increasing release of endogenous glutamate through reversal of Na-dependant glutamate transport (Stys PK, 2005). The observed changes support an improvement of glutamate neurotransmission.

Although patients were slightly more tired with moderate decrease in clinical performance after bloodletting, rhEPO induced a major clinical improvement in terms of fatigue but also several clinical features related to cognitive functions.

Interestingly, neurophysiological changes were already observed after the performance of bloodletting before treatment with exogenous erythropoietin, suggesting that either iron regulation alone or endogenous erythropoietin production related to anemia could improve the physiology of this neurotransmitter.

Glutamate physiology is dependent on the presence and disposability of energy, suggesting that the changes induced by the successive bloodlettings are related to an increased local production of energy in the cortex.

Neurodegeneration due to lack of energy is resumed in the following cascade of events. A number of factors (nitric oxide; ischemia that results from inflammation of small blood vessels; and decreased expression of genes that encode mitochondrial redox carriers) contribute to energy failure and subsequent rundown of the Na+, K+-ATPase pump, with subsequent depolarization and loss of capacity to maintain transmembrane ion gradients. The depolarization activates sodium channels (e.g., Nav1.6), which provides a route for persistent sodium influx. This process, in turn, drives the Na+/Ca2+ exchanger to operate in a calcium-importing mode. The rise in intracellular calcium induces a further increase in calcium levels via calcium-induced calcium release. Increased intra-axonal calcium also injures mitochondria, and activates nitric oxide synthase and harmful proteases and lipases (for review see Waxman). One of the potential effects of EPO is through an improvement of the Na+/K+ ATPase pump in the central nervous system as it has been shown in cardiac muscle.

It is important to note that the combination of iron deprivation through bloodletting associated with recombinant erythropoietin results in synergic effects which cannot be obtained from isolated processes.

The above results demonstrate that the first step of iron deprivation improves cortical excitability and in one patient clinical scales. The second step of the process, using recombinant erythropoietin, largely increases the results obtained.

The literature showed that iron deprivation does not improve MS (Lynch et al., 2000). However, the inventors induced an iron deprivation until induction of anaemia which resulted in production of endogenous erythropoietin which can be obtained only if the deprivation is sufficiently severe to induce anaemia.

The second step of the process with recombinant erythropoietin can be performed with sufficient security only if it is associated with an iron deprivation which precludes the normal erythropoiesis. Indeed, an excellent tolerance of the treatment was observed without production of an excess of red blood cells and without requiring subsequent bloodletting. The approach that has been performed by Ehrenreich (2007) in patients treated by erythropoietin was completely different. Indeed, bloodletting was only performed to reduce the number of red blood cells induced by the recombinant erythropoietin. Ehrenreich et al. observed clinical and electrophysiological improvement only in patients receiving high dose of erythropoietin (48,000 IU/week). No improvement was observed in patients receiving 8,000 to 16,000 IU/week. The treatment by erythropoietin performed as described by Ehrenreich et al. is associated with a higher risk of thrombosis resulting from the increase of the number of red blood cells and haematocrit. To the contrary, the iron deprivation that has been performed by the inventors precludes any risk resulting from the effect of erythropoietin. The above study also illustrates that iron deprivation allows to treat through erythropoietin without subsequent requirements of bloodletting during several months. Moreover, the association of iron deprivation and erythropoietin combines two effects, the first one being anti-inflammatory effect from iron deprivation and the second one being the neuroprotective effect by erythropoietin. This may explain why the inventors observed clinical and biological improvements in patients receiving no more than 20,000 IU/week of erythropoietin.

This combined treatment can be proposed to conditions with similar physiopathological processes. This is the case for chronic inflammatory demyelinating polyneuropathies. Chronic inflammatory demyelinating polyneuropathies are characterized by inflammation, demyelination, progressive axonal loss resulting from energy expenses that exceed the capacity of the nerve and result in Ca++ induced neurodegeneration. The present innovation can hence be similarly proposed to this condition.

### CONCLUSION

The above study demonstrates several findings:
Improvement of axonal physiology of multiple sclerosis through (1) erythropoietin-induced secretion by bloodletting and iron deficit, and (2) association of rhEPO after iron depletion.
Improvement of several clinical parameters including: fatigue, cognitive functions, walking distance, walking speed, through (1) erythropoietin-induced secretion by bloodletting and iron deficit, and (2) association of rhEPO after iron depletion.
Tolerance of the two-step process of iron depletion followed by exogenous rhEPO on biological and clinical parameters.

### REFERENCES

Abo-Krysha, N. and Rashed, L. (2008) The role of iron dysregulation in the pathogenesis of multiple sclerosis: an Egyptian study. Mult Scler, 14, 602-608.
Adembri, C., Massagrande, A., Tani, A., Miranda, M., Margheri, M., De Gaudio, R. and Pellegrini-Giampietro, D. E. (2008) Carbamylated erythropoietin is neuroprotective in an experimental model of traumatic brain injury. Crit Care Med, 36, 975-978.
Bergeron, R. J., Wiegand, J., Dionis, J. B., Egli-Karmakka, M., Frei, J., Huxley-Tencer, A. and Peter, H. H. (1991) Evaluation of desferrithiocin and its synthetic analogues as orally effective iron chelators. J Med Chem, 34, 2072-2078.
Bianchi, R., Buyukakilli, B., Brines, M., Savino, C., Cavaletti, G., Oggioni, N., Lauria, G., Borgna, M., Lombardi, R., Cimen, B., Comelekoglu, U., Kanik, A., Tataroglu, C., Cerami, A. and Ghezzi, P. (2004) Erythropoietin both protects from and reverses experimental diabetic neuropathy. Proc Natl Acad Sci USA, 101, 823-828.
Boesch, S., Sturm, B., Hering, S., Scheiber-Mojdehkar, B., Steinkellner, H., Goldenberg, H. and Poewe, W. (2008) Neurological effects of recombinant human erythropoietin in Friedreich's ataxia: a clinical pilot trial. Mov Disord, 23, 1940-1944.
Bokemeyer, C., Aapro, M. S., Courdi, A., Foubert, J., Link, H., Osterborg, A., Repetto, L. and Soubeyran, P. (2007) EORTC guidelines for the use of erythropoietic proteins in anaemic patients with cancer: 2006 update. Eur J Cancer, 43, 258-270.
Corwin, H. L., Gettinger, A., Fabian, T. C., May, A., Pearl, R. G., Heard, S., An, R., Bowers, P. J., Burton, P., Klausner, M. A. and Corwin, M. J. (2007) Efficacy and safety of epoetin alfa in critically ill patients. N Engl J Med, 357, 965-976.
Ehrenreich, H., Aust, C., Krampe, H., Jahn, H., Jacob, S., Herrmann, M. and Siren, A. L. (2004) Erythropoietin: novel approaches to neuroprotection in human brain disease. Metab Brain Dis, 19, 195-206.
Ehrenreich, H., Fischer, B., Norra, C., Schellenberger, F., Stender, N., Stiefel, M., Siren, A. L., Paulus, W., Nave, K. A., Gold, R. and Bartels, C. (2007) Exploring recombinant human erythropoietin in chronic progressive multiple sclerosis. Brain, 130, 2577-2588.
Ehrenreich, H., Hasselblatt, M., Dembowski, C., Cepek, L., Lewczuk, P., Stiefel, M., Rustenbeck, H. H., Breiter, N., Jacob, S., Knerlich, F., Bohn, M., Poser, W., Ruther, E., Kochen, M., Gefeller, O., Gleiter, C., Wessel, T. C., De Ryck, M., Itri, L., Prange, H., Cerami, A., Brines, M. and Siren, A. L. (2002) Erythropoietin therapy for acute stroke is both safe and beneficial. Mol Med, 8, 495-505.
Forge, J. K., Pedchenko, T. V. and LeVine, S. M. (1998) Iron deposits in the central nervous system of SJL mice with experimental allergic encephalomyelitis. Life Sci, 63, 2271-2284.
Gene, K., Genc, S., Baskin, H. and Semin, I. (2006) Erythropoietin decreases cytotoxicity and nitric oxide formation induced by inflammatory stimuli in rat oligodendrocytes. Physiol Res, 55, 33-38.
Genc, S., Koroglu, T. F. and Genc, K. (2004) Erythropoietin and the nervous system. Brain Res, 1000, 19-31.
Hartley, C. E., Varma, M., Fischer, J. P., Riccardi, R., Strauss, J. A., Shah, S., Zhang, S, and Yang, Z. J. (2008) Neuroprotective effects of erythropoietin on acute metabolic and pathological changes in experimentally induced neurotrauma. J Neurosurg, 109, 708-714.
Hobart, J. C., Riazi, A., Lamping, D. L., Fitzpatrick, R. and Thompson, A. J. (2003) Measuring the impact of MS on walking ability: the 12-Item MS Walking Scale (MSWS-12). Neurology, 60, 31-36.
Keswani, S. C., Buldanlioglu, U., Fischer, A., Reed, N., Polley, M., Liang, H., Zhou, C., Jack, C., Leitz, G. J. and Hoke, A. (2004) A novel endogenous erythropoietin mediated pathway prevents axonal degeneration. Ann Neurol, 56, 815-826.
Khwaja, O. and Volpe, J. J. (2008) Pathogenesis of cerebral white matter injury of prematurity. Arch Dis Child Fetal Neonatal Ed, 93, F153-161.
Krupp, L. B., LaRocca, N. G., Muir-Nash, J. and Steinberg, A. D. (1989) The fatigue severity scale. Application to patients with multiple sclerosis and systemic lupus erythematosus. Arch Neurol, 46, 1121-1123.
Kurtzke, J. F. (1983) Rating neurologic impairment in multiple sclerosis: an expanded disability status scale (EDSS). Neurology, 33, 1444-1452.
Li, W., Maeda, Y., Yuan, R. R., Elkabes, S., Cook, S, and Dowling, P. (2004) Beneficial effect of erythropoietin on experimental allergic encephalomyelitis. Ann Neurol, 56, 767-777.
Lublin, F. D. and Reingold, S. C. (1996) Defining the clinical course of multiple sclerosis: results of an international survey. National Multiple Sclerosis Society (USA) Advisory Committee on Clinical Trials of New Agents in Multiple Sclerosis. Neurology, 46, 907-911.
Lynch, S. G., Fonseca, T. and Levine, S. M. (2000) A multiple course trial of desferrioxamine in chronic progressive multiple sclerosis. Cell Mol Biol (Noisy-le-grand), 46, 865-869.
Lynch, S. G., Peters, K. and LeVine, S. M. (1996) Desferrioxamine in chronic progressive multiple sclerosis: a pilot study. Mult Scler, 2, 157-160.
Macdougall, I. C. (2008) Novel erythropoiesis-stimulating agents: a new era in anemia management. Clin J Am Soc Nephrol, 3, 200-207.
Meijler, M. M., Arad-Yellin, R., Cabantchik, Z. I. and Shanzer, A. (2002) Synthesis and evaluation of iron chelators with masked hydrophilic moieties. J Am Chem Soc, 124, 12666-12667.
Mitchell, K. M., Dotson, A. L., Cool, K. M., Chakrabarty, A., Benedict, S. H. and LeVine, S. M. (2007) Deferiprone, an orally deliverable iron chelator, ameliorates experimental autoimmune encephalomyelitis. Mull Scler, 13, 1118-1126.
Qureshi, S. A., Kim, R. M., Konteatis, Z., Biazzo, D. E., Motamedi, H., Rodrigues, R., Boice, J. A., Calaycay, J. R., Bednarek, M. A., Griffin, P., Gao, Y. D., Chapman, K. and Mark, D. F. (1999) Mimicry of erythropoietin by a nonpeptide molecule. Proc Natl Acad Sci USA, 96, 12156-12161.
Siren, A. L., Fratelli, M., Brines, M., Goemans, C., Casagrande, S., Lewczuk, P., Keenan, S., Gleiter, C., Pasquali, C., Capobianco, A., Mennini, T., Heumann, R., Cerami, A., Ehrenreich, H. and Ghezzi, P. (2001) Erythropoietin prevents neuronal apoptosis after cerebral ischemia and metabolic stress. Proc Natl Acad Sci USA, 98, 4044-4049.
Stys P K, W. S. (2005) Molecular mechanisms of calcium influx in axonal degeneration. In: Waxman SG, ed. Multiple sclerosis as a neuronal disease. San Diego: Elsevier Academic Press, 275-292.
Toth, C., Martinez, J. A., Liu, W. Q., Diggle, J., Guo, G. F., Ramji, N., Mi, R., Hoke, A. and Zochodne, D. W. (2008) Local erythropoietin signaling enhances regeneration in peripheral axons. Neuroscience, 154, 767-783.
Wiese, L., Hempel, C., Penkowa, M., Kirkby, N. and Kurtzhals, J. A. (2008) Recombinant human erythropoietin increases survival and reduces neuronal apoptosis in a murine model of cerebral malaria. Malar J, 7, 3.

1. A kit of parts comprising, in separate containers, an erythropoiesis-stimulating agent (ESA) and an iron chelator.

2. The kit of claim 1, further containing a notice of use which indicates at least that the kit can be used for the treatment of multiple sclerosis.

3. A pharmaceutical composition comprising an ESA and an iron chelator.

4. The kit of claim 1, or the pharmaceutical composition of claim 3, wherein said iron chelator is selected in the group consisting of deferiprone, deferoxamine, polyanionic amines, substituted polyaza compounds, desferrithiocon, hydroxybenzyl-ethylenediamine-diacetic acid and pyridoxal isonicotinoyl hydrazone.

5. The kit of claim 1, or the pharmaceutical composition of claim 3, wherein said ESA is recombinant human erythropoietin.

6. The kit of claim 1, or the pharmaceutical composition of claim 3, wherein said ESA is selected in the group consisting of darbopoietin alpha, CERA and Hematide.

7. A method of treating multiple sclerosis comprising administering the pharmaceutical composition of claim 3.

8. The method of claim 7, wherein said iron chelator is administered to render or maintain a patient depleted in iron.

9. The method of claim 7, wherein said ESA is administered to a patient depleted in iron.

10. The method of claim 7, wherein said iron chelator is administered before said ESA.

11. A method of treating multiple sclerosis in a patient depleted in iron comprising administering an ESA.

12. The method of claim 7, wherein said ESA is recombinant human erythropoietin.

13. The method of claim 7, wherein said ESA is selected in the group consisting of darbopoietin alpha, CERA and Hematide.

14. The method of claim 7, wherein said iron chelator is selected in the group consisting of deferiprone, deferoxamine, polyanionic amines, substituted polyaza compounds, desferrithiocon, hydroxybenzyl-ethylenediamine-diacetic acid and pyridoxal isonicotinoyl hydrazone.

15. A method of inducing an anaemic state in a patient suffering from multiple sclerosis comprising administering an iron chelator in an amount sufficient to induce the anaemic state.

16. The method of claim 15, wherein the patient also receiving an ESA.

17. The method according to claim 15 wherein the iron chelator is selected in the group consisting of deferiprone, deferoxamine, polyanionic amines, substituted polyaza compounds, desferrithiocon, hydroxybenzyl-ethylenediamine-diacetic acid and pyridoxal isonicotinoyl hydrazone.
